(19) United States
(12) Patent Application Publication
(10) Pub. No.: US 2024/0050742 A1
(43) Pub. Date: Feb. 15, 2024

(54) STIMULATION PATTERNS FOR THERAPY

(71) Applicant: Medtronic, Inc., Minneapolis, MN (US)

(72) Inventors: Lisa M. Jungbauer Nikolas, Lino Lakes, MN (US); Sarah J. Offutt, Golden Valley, MN (US); Katie C. Bittner, White Bear Lake, MN (US); Julia P. Slopsema, Minneapolis, MN (US); Thaddeus S. Brink, St. Paul, MN (US); Leonid M. Litvak, Bet Shemesh (IL)

(21) Appl. No.: 18/446,598

(22) Filed: Aug. 9, 2023

Related U.S. Application Data
(60) Provisional application No. 63/371,157, filed on Aug. 11, 2022.

(51) Int. Cl. A61N 1/36

(52) U.S. Cl. CPC A61N 1/36007; A61N 1/36178; A61N 1/36175; A61N 1/3615; A61N 1/36062

(57) ABSTRACT

Methods, systems, and devices are configured delivering one or more sequences of different pulse trains to a patient. For example, a system includes processing circuitry configured to control stimulation circuitry to deliver a sequence of a plurality of trains of electrical stimulation pulses, wherein each train of the plurality of trains of electrical stimulation pulses comprises respective pulses at least partially defined by a unique parameter variation pattern of a plurality of parameter variation patterns, and control the stimulation circuitry to repeatedly deliver the sequence of the plurality of trains of electrical stimulation pulses.

This application claims the benefit of U.S. Provisional Patent Application No. 63/371,157, filed Aug. 11, 2022, the entire contents of which is incorporated herein by reference.

### TECHNICAL FIELD

The disclosure relates to medical devices and, more particularly, medical devices that deliver therapy to a patient.

### BACKGROUND

Bladder dysfunction, such as overactive bladder, urgency, or urinary incontinence, are problems that may afflict people of all ages, genders, and races. Various muscles, nerves, organs and conduits within the pelvic floor cooperate to collect, store and release urine. A variety of disorders may compromise urinary or bowel function, and contribute to an overactive bladder, urgency, urinary incontinence, urinary retention, fecal incontinence, or other conditions. Many of the disorders may be associated with aging, injury, or illness.

Urinary incontinence may include urge incontinence, urinary frequency, and stress incontinence. In some examples, urge incontinence may be caused by disorders of peripheral or central nervous systems that control bladder micturition reflexes. Some patients may also suffer from nerve disorders that prevent proper triggering and operation of the bladder, sphincter muscles, or nerve disorders that lead to overactive bladder activities or urge incontinence.

Urinary incontinence can be attributed to improper sphincter function, either in the internal urinary sphincter or external urinary sphincter. For example, aging can result in weakened sphincter muscles, which may cause incontinence. Nerves running though the pelvic floor stimulate contractility in the sphincter. An improper communication between the nervous system and the urethra or urinary sphincter can result in a bladder dysfunction, such as overactive bladder, urgency, urge incontinence, or another type of urinary incontinence. Nerve disorders, weakened bladder muscles, or obstructions of a urethra may lead urinary retention, in which the patient is unable to empty their bladder completely. Other conditions may include idiopathic bladder dysfunction in which there is an improper communication between the brain and the bladder which can lead to overactive bladder, urgency, and other conditions.

### SUMMARY

In general, the disclosure is directed to techniques, devices, and/or systems for treating various pelvic floor disorders, such us bladder dysfunction (e.g., urge, frequency, or stress incontinence) or bowel dysfunction. For example, a system may deliver electrical stimulation that has a sequence of different trains of pulses, and the system may repeat the sequence over time. Some, or all, of the different trains of pulses may have pulses that vary according to a unique parameter variation pattern. A parameter variation pattern may define how one or more parameters are varied between the pulses within the train. For example, one train may have pulses that are delivered by two or more different electrode combinations, and another train may have pulses that have different amplitudes. The system may then deliver a repeatable sequence that includes multiple trains of pulses wherein some or all of the trains have unique parameter variation patterns.

In one example, a method includes a system including processing circuitry configured to control stimulation circuitry to deliver a sequence of a plurality of trains of electrical stimulation pulses, wherein each train of the plurality of trains of electrical stimulation pulses comprises respective pulses at least partially defined by a unique parameter variation pattern of a plurality of parameter variation patterns; and control the stimulation circuitry to repeatedly deliver the sequence of the plurality of trains of electrical stimulation pulses.

In another example, a system includes a method including controlling, by processing circuitry, stimulation circuitry to deliver a sequence of a plurality of trains of electrical stimulation pulses, wherein each train of the plurality of trains of electrical stimulation pulses comprises respective pulses at least partially defined by a unique parameter variation pattern of a plurality of parameter variation patterns; and controlling, by the processing circuitry, the stimulation circuitry to repeatedly deliver the sequence of the plurality of trains of electrical stimulation pulses.

In another example, a computer readable storage medium includes instructions that, when executed, causes processing circuitry to control stimulation circuitry to deliver a sequence of a plurality of trains of electrical stimulation pulses, wherein each train of the plurality of trains of electrical stimulation pulses comprises respective pulses at least partially defined by a unique parameter variation pattern of a plurality of parameter variation patterns; and control the stimulation circuitry to repeatedly deliver the sequence of the plurality of trains of electrical stimulation pulses.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is conceptual diagram illustrating an example system configured to deliver electrical stimulation to a patient, in accordance with the examples of this disclosure.

FIG. 2 is a conceptual block diagram illustrating an example of an IMD configured to deliver therapy to a patient.

FIG. 3 is a block diagram illustrating an example external programmer.

FIG. 4A is a conceptual diagram of an example lead and electrode combinations.

FIGS. 4B, 4C, 4D, 4E, 4F, 4G, 4H, and 4I are timing diagrams of example parameter patterns for trains of electrical stimulation pulses.

FIG. 5 is a flow diagram illustrating an example technique for delivering electrical stimulation having a sequence of different trains of pulses.

### DETAILED DESCRIPTION

The disclosure is directed to techniques, systems, and devices configured to deliver electrical stimulation to a patient, such as electrical stimulation configured to treat conditions associated with the pelvic floor (e.g., bladder dysfunction or bowel dysfunction). In some examples, a system may be configured to deliver a sequence of pulse trains, wherein at least some of the different pulse trains are one or more parameters that vary according to a unique parameter variation pattern. Bladder dysfunction generally refers to a condition of improper functioning of the bladder or urinary tract, and may include, for example, an overactive bladder, urgency, urgency frequency, bladder incontinence, urinary incontinence, urgency frequency, and/or urinary retention. Urgency is a sudden, compelling urge to urinate, and may often, though not always, be associated with urinary incontinence. Overactive bladder may include excessive contractions of the detrusor muscle (e.g., smooth muscle residing in the wall of the bladder) and may be one of the causes for urgency. Urinary incontinence refers to a condition of involuntary loss of urine, and may include urge incontinence, stress incontinence, or both stress and urge incontinence, which may be referred to as mixed urinary incontinence. As used in this disclosure, the term “urinary incontinence” includes disorders in which urination occurs when not desired, such as stress or urge incontinence (e.g., overactive bladder). Urinary retention, such as non-obstructive urinary retention, refers to a condition in which a patient is unable to empty their bladder completely. Bowel disorders can also include various dysfunction of the bowel that manifests as fecal incontinence, intractable constipation, irritable bowel syndrome, or inflammatory bowel disease for example. Additional pelvic floor disorders include neurogenic bowel/bladder (tremor, Parkinson's disease, epilepsy, multiple sclerosis, stork, spinal cord injury, neuropathy etc.), sexual dysfunction, obesity, gastroparesis, pelvic pain, chronic pain, and interstitial cystitis.

In order to void urine, the nervous system and several muscles of the body typically work in concert to expel urine from the bladder. For example, the internal urinary sphincter muscle and the external urinary sphincter muscle relax to allow urine to pass through the openings in these sphincters. In addition, the detrusor muscle in the wall of the bladder contracts to increase the internal bladder pressure and force urine out of the bladder and through the urethra and past the urinary sphincters. Bladder dysfunction can occur when portions of the nervous system that innervate these muscles, or the muscles themselves, impede the voluntary or involuntary mechanisms either preventing the patient from retaining urine until the patient voluntarily decides to urinate or leading to incomplete emptying of the bladder (retention).

For example, urge incontinence may be caused by dysfunction of peripheral or central nervous systems that control bladder micturition reflexes. Some patients may also suffer from nerve disorders or unknown issues that prevent proper triggering and operation of the bladder (which may include the detrusor muscle), sphincter muscles or nerve disorders that lead to overactive bladder activities or urge incontinence. Additionally, or alternatively, urinary incontinence can be attributed to improper sphincter function, either in the internal urinary sphincter or external urinary sphincter. In some examples, aging can result in weakened sphincter muscles, which may cause incontinence. Nerves running though the pelvic floor stimulate contractility in the sphincter. An improper communication between the nervous system and the urethra or urinary sphincters can result in a bladder dysfunction, such as overactive bladder, urgency, urge incontinence, urgency frequency, urinary retention, or another type of urinary condition.

Delivery of electrical stimulation can lead to activation and/or suppression of neural and muscular activities, resulting in a therapeutic effect. For example, electrical stimulation can be delivered to the sacral nerve of the patient to reduce symptoms associated with bladder or bowel incontinence. However, continuous electrical stimulation of the sacral nerve requires more power than may be necessary to treat the patient, whereas reduced duration and/or power usage for stimulation can result in extended battery life of an implanted medical device (IMD) that delivers the electrical stimulation. In addition, or alternatively, continuous delivery of electrical stimulation pulses having the same parameters may reduce the sensitivity (e.g. reduce the responsiveness) of the nervous system to the delivered stimulation over time, a phenomenon that is sometimes referred to as habituation or accommodation (e.g., a reduction in the efficacy of stimulation over time). Habituation may result in less effective stimulation and a decrease in therapeutic efficacy of the stimulation. Some frequency or pulse width values, for example, of stimulation pulses may be more susceptible to habituation than other values for those parameters. When the therapy efficacy decreases, the patient may experience a reduced quality of life, the patient may abandon therapy, the patient may resort to other therapy modalities (e.g., medication), and/or a clinician may need to intervene to find alternative stimulation parameters or electrode configurations that may restore therapy efficacy at previously expected levels.

As described in this disclosure, various techniques and systems may be used to deliver electrical stimulation using pulses that vary in one or more parameter values over time. For example, an IMD may be configured to deliver a sequence of different pulse trains over time. The IMD may repeat this sequence as needed to continue delivering therapy. The sequence of pulse trains may include two or more pulse trains, where each pulse train includes a plurality of pulses. At least two of the pulse trains of the sequence, up to all of the pulse trains of the sequence, may have pulses that are varied by a unique parameter variation pattern.

The parameter variation patterns may be unique in that the unique parameter variation patterns within a sequence are different from each other in at least one aspect, such as variation of a different parameter or a different type of variation for the same parameter. Example parameter variation patterns include varying the electrode combination used to deliver the pulses within a pulse train, varying the amplitude of pulses within the pulse train, varying the frequency of pulses within the pulse train, varying the pulse width of pulses within the pulse train, varying the type of recharge phase in the pulse train (e.g., pulses that include a passive recharge phase or active recharge phase), varying an inter-pulse interval within the pulse train (e.g., which may manifest as one or more bursts of pulses within the pulse train), varying a pulse shape of the pulses within the pulse train, or any other variation of any other parameter that at least partially defines the pulses of the pulse train. In some examples, a parameter variation pattern may include variations to two or more parameters within a single pulse train (e.g., varying an amplitude and electrode combination for the pulses within one pulse train).

The IMD may insert an inter-train, or inter-program delay between the delivery of the pulse trains of the sequence. This delay may be a period of time that is the same between all pulse trains or different for different pulse trains. In addition, the IMD may repeat the sequence of pulse trains over time. In some examples, the IMD may insert a delay between the last pulse train of the sequence and the first pulse train of the sequence when it is repeated. The IMD may also change the pulse trains of the sequence over time. For example, the IMD or programmer may remove a pulse train in response to sensed information indicating the pulse train is ineffective or user input requesting that the pulse train no longer be used. The IMD may continue to deliver the sequence without the removed pulse train or replace the removed pulse train with a different pulse train that may have a different parameter variation pattern. In some examples, the IMD or programmer may add additional pulse trains with different parameter variation patterns in response to user requests or sensed information indicating that such change to stimulation may be beneficial. The IMD and/or programmer may also change the duty cycle of one or more trains within the sequence or the sequence as a whole in order to improve therapeutic efficacy and/or reduce power consumption.

The electrical stimulation described herein may provide one or more advantages. For example, delivering a sequence of pulse trains that include different parameter variation patterns may enable the system to reduce the overall delivery of stimulation and reduce power consumption of the IMD. In addition, delivering a sequence of pulse trains that include different parameter variation patterns may reduce or eliminate habituation to allow the therapy to be effective for the patient for longer periods of time. For example, delivering a new pattern of pulses may re-establish a therapeutic effect in the patient. In this manner, the patient may need to less frequently adjust stimulation, and the clinician may need to spend less time adjusting therapy over time for the patient. Other therapeutic and system benefits may also be achieved by the examples described herein.

Implantable medical devices are generally described herein, but the stimulation systems and techniques described herein may utilize chronic neurostimulators or trial neurostimulators that are external, partially implantable, or fully implantable. Chronic neurostimulators are usually implanted and are intended to provide long term therapy (e.g. 5-20 years). For sacral neuromodulation, chronic implants typically include a battery that is connected to a lead with electrodes. Trial neurostimulators are temporary neurostimulators to determine if the patient will be responsive to treatment. Trial neurostimulators are usually an external battery/stimulator that couples to an implanted lead (e.g. a temporary lead), but may also be implanted or partially implanted. Although implantable devise are described herein for illustration, external medical devices may be used in other examples according to the techniques described herein.

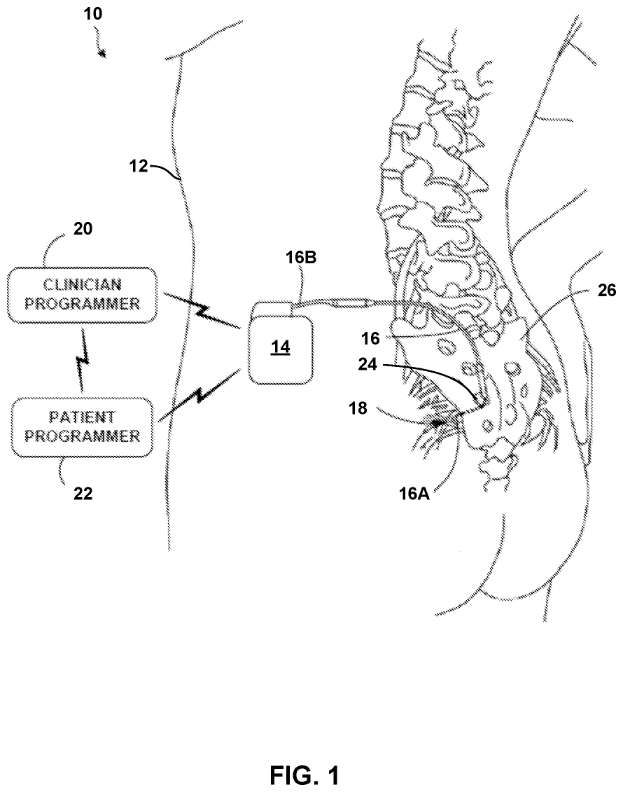

FIG. 1 is conceptual diagram illustrating an example system 10 configured to deliver stimulation therapy to a patient 12 using one or more sequences of different pulse trains as described herein. Patient 12 ordinarily will be a human patient. In some cases, however, system 10 may be applied to other mammalian or non-mammalian, non-human patients. In some examples, patient 12 experiences bladder dysfunction, such as, for example, improper functioning of a bladder, a urinary sphincter, or a urinary tract, and may include an overactive bladder, urgency, urinary incontinence, urgency frequency, urinary retention, or combinations thereof. In some examples, the systems and techniques described herein may also be applied to the delivery of stimulation to nerves and/or muscles in order to treat bowel disfunction such as fecal incontinence or other disorders.

System 10 is configured to deliver stimulation that includes a sequence of different trains of pulses. Each train of pulses within the sequence may be different by at least one parameter value from the pulses of another train. The parameter value indicates the value of a particular parameter, such as which electrodes are configured to deliver stimulation, a magnitude of current amplitude, a period of time for a pulse width, a value for pulse frequency, etc. In other words, each parameter indicates a characteristic that at least partially defines stimulation, such as current amplitude, voltage amplitude, pulse width, pulse frequency, pulse shape, inter-pulse duration, electrode combination, polarity, pulse burst duration, etc. Each parameter then has a value that can be varied to define that particular parameter for a specific train of pulses. In this manner, variation of a parameter indicates the variation of a value for that particular parameter. A unique parameter variation pattern may specify how one or more parameters are varied within a train of pulses. In one example, a first train of pulses of a sequence may have pulses that are delivered by two or more different electrode combinations. A second train of the same sequence may have pulses that have different amplitudes. System 10 can then repeat this sequence (of two or more different pulse trains with different unique parameter variation patterns) of pulse trains over a period of time to the patient.

In this manner, system 10 can be configured to deliver stimulation therapy using a repeating sequence of different trains of stimulation pulses. For example, clinician programmer 20, patient programmer 22, or IMD 14 may be configured to control delivery of a therapy to patient 12 based on a defined sequence of pulse trains. Examples of different pulse trains are described in more detail below. While FIG. 1 illustrates an implantable medical device (IMD 14), it is understood that concepts disclosed herein may apply to external or trial neurostimulators.

IMD 14 may provide electrical stimulation therapy to target tissue site 18 located proximate a sacral nerve, a pudendal nerve, a hypogastric nerve, a pelvic nerve, tibial nerve, saphenous nerve, or another nerve associated with the bladder of patient 12 by generating a programmable electrical stimulation signal (e.g., in the form of electrical pulses) and delivering the electrical stimulation signal to target tissue site 18 via lead 16. In some examples, lead 16 includes one or more stimulation electrodes disposed on the distal end 16A of lead 16 and implanted proximate to target tissue site 18 such that the electrical stimulation is delivered from IMD 14 to target tissue site 18 via the stimulation electrodes. The electrical stimulation therapy may be used to treat bladder dysfunction of patient 12.

In general, the sacral nerves include five sacral nerves that emerge from the sacrum. In some examples, the sacral vertebrae (S1-S5) may be used to number the sacral nerves. The sacral nerves contribute to the sacral plexus (a network of intersecting nerves that innervates the posterior thigh, part of the lower leg, the foot, and part of the pelvis) and the coccygeal plexus (a network of intersecting nerves near the coccyx bone, e.g., the tailbone, that innervates the skin of the coccyx bone and around the anus). In general, the pudendal nerve is a somatic nerve in the pelvic region, which is a large branch of the sacral plexus. The pudendal nerve innervates the external genitalia, the urinary sphincters, and the anal sphincters. The hypogastric nerves and pelvic nerves innervate the detrusor muscle of the bladder. Stretch receptors of the bladder may send signals to the brain of patient 12 via afferent sensory fibers in sacral nerves that may result in increased activity of the detrusor muscle of the bladder.

As illustrated in the example of FIG. 1, distal end 16A of lead 16 is implanted proximate to target tissue site 18. In the example shown in FIG. 1, target tissue site 18 is proximate the S3 sacral nerve of patient 12. In this example, in order to implant the distal end 16A of lead 16 proximate to the S3 sacral nerve, lead 16 may be introduced into the S3 sacral foramen 24 of sacrum 26 to access the S3 sacral nerve. For some patients, stimulation of the S3 sacral nerve may be effective in treating bladder dysfunction of patient 12. In other examples, distal end 16A may be implanted proximate to a different target tissue site, such as a target tissue site proximate to a different sacral nerve, a pudendal nerve, a hypogastric nerve, a pelvic nerve, an afferent nerve, or another nerve associated with the bladder of patient 12 to treat the bladder dysfunction of patient 12. Distal end 16A of lead 16 may include multiple electrodes, such as four or more electrodes.

Although FIG. 1 illustrates one lead 16, in some examples, IMD 14 may be coupled to two or more leads, e.g., to facilitate bilateral (e.g., stimulation to sacral nerves on either side of the spinal cord) or multi-lateral stimulation (e.g., stimulation to multiple sacral nerves on the same side of the spinal cord). In some examples, lead 16 may also carry one or more sense electrodes via which IMD 14 can sense one or more physiological parameters (e.g., nerve signals, EMG, or the like) of patient 12, in addition to the one or more stimulation electrodes carried by lead 16. In other examples, the same electrodes used to stimulate may also be used for sensing. In some examples, lead 16 includes a lead body, and proximal end 16B of lead 16 may be electrically coupled to IMD 14 via one or more conductors extending substantially through the lead body between the one or more stimulation electrodes carried by lead 16 and IMD 14. In the case of an external device, proximal end 16B may be configured to couple to an external medical device, such as trial neurostimulator.

In the example shown in FIG. 1, lead 16 is cylindrical. One or more electrodes of lead 16 may be ring electrodes, segmented electrodes, or partial ring electrodes. Segmented or partial ring electrodes each extend along an arc less than 360 degrees (e.g., 70-120 degrees) around the outer perimeter of the lead 16, where in some example, multiple electrode segments are disposed around the perimeter of lead 16 at the same axial position of lead 16. In some examples, segmented electrodes may be useful for targeting different fibers of the same or different nerves to generate different physiological effects or for delivering relatively higher frequency stimulation (e.g., about 66 Hertz) and relatively lower frequency stimulation (e.g., about 15 Hertz) to activate both fast twitch muscles and slow twitch muscles substantially simultaneously or at alternating time slots. In some examples, different pulse width values may be selected in order to selectively stimulate certain nerve fibers while avoiding other nerve fibers (e.g., fibers having different diameters). In some examples, lead 16 may include a paddle-shaped (e.g., a “paddle” lead) portion with a flat or curved surface.

In some examples, one or more of the electrodes of lead 16 may be cuff electrodes that are configured to extend at least partially around a nerve (e.g., extend axially around an outer surface of a nerve). In some cases, delivering stimulation via one or more cuff electrodes and/or segmented electrodes may help achieve a more uniform electrical field or activation field distribution relative to the nerve in some examples, which may help minimize discomfort to patient 12 that results from the delivery of electrical stimulation. An electrical field represents the areas of a patient anatomical region that are covered by an electrical field during delivery of electrical stimulation to tissue within patient 12. The electrical field may define the volume of tissue that is affected when the electrodes of lead 16, or any other electrodes carried by IMD 14 (e.g., one or more electrodes carried by the housing of IMD 14), are activated. An activation field represents the neurons that will be activated by the electrical field in the neural tissue proximate to the activated electrodes.

The illustrated numbers and configurations of lead 16 and electrodes carried by lead 16 are merely one example. Different configurations, e.g., different quantities and/or positions of leads and electrodes, are possible. In other examples, IMD 14 may be coupled to additional leads or lead segments having one or more electrodes positioned at different locations in the pelvic region of patient 12. In some examples, lead 16 may have one electrode, two electrodes, three electrodes, four electrodes, or eight electrodes. In other examples, lead 16 may have a combination of ring electrodes and segmented electrodes.

IMD 14 may be surgically implanted in patient 12 at any suitable location within patient 12, such as within in an abdomen of patient 12. In some examples, the implantation site may be a subcutaneous location in the upper buttocks, side of the lower abdomen, or the side of the lower back. IMD 14 has a biocompatible outer housing, which may be formed from titanium, stainless steel, a liquid crystal polymer, or the like. In some examples, such as with a leadless stimulator, the outer housing may have an electrically insulative coating (e.g., parlyene). In some examples, electrical conductors disposed within the lead body of lead 16 electrically connect electrodes to an electrical stimulation delivery module within IMD 14. In other examples, system 10 may include a leadless electrical stimulator, such as a microstimulator (e.g., a capsule shaped microstimulator), where the leadless electrical stimulator delivers electrical stimulation to target tissue site 18.

In the example illustrated in FIG. 1, system 10 includes clinician programmer 20 and patient programmer 22. In some examples, one or both programmers 20 and 22 may be wearable communication devices integrated into a remote, key fob, or a wrist watch. In other examples, one or both programmers 20 and 22 may be handheld computing devices, computer workstations, smartphones, personal computers, or networked computing devices. Programmers 20 and/or 22 may be proprietary devices or computing devices configured to execute different programs, one or more of which may include an application that, when executed, causes the programmer to function as described herein. Programmers 20 and 22 may include respective user interfaces that receive input from a user (e.g., a clinician or patient 12, respectively). The user interfaces may include components for interaction with a user, such as a keypad and a display. In some examples, the display may be a cathode ray tube (CRT) display, a liquid crystal display (LCD), or light emitting diode (LED) display and the keypad may take the form of an alphanumeric keypad or a reduced set of keys associated with particular functions. Programmers 20 and 22 can, additionally or alternatively, include a peripheral pointing device, e.g., a mouse, via which a user may interact with the user interface. In some examples, the displays may include a touch screen display, and a user may interact with programmers 20 and 22 via the touch screens of the displays. In some examples, the user may also interact with programmers 20 and 22 and/or IMD 14 remotely via a networked computing device.

Clinician programmer 20 facilitates interaction of a clinician with one or more components of system 10. In some examples, the clinician, (e.g., physician, technician, surgeon, electrophysiologist, or other clinician) may interact with clinician programmer 20 to communicate with IMD 14. For example, the clinician may retrieve physiological or diagnostic information from IMD 14 via clinician programmer 20. As another example, the clinician may interact with programmer 20 to program IMD 14, e.g., select values that define electrical stimulation generated and delivered by IMD 14, select other operational parameters of IMD 14, or the like. For example, as described herein, a clinician may select variations of parameters, which pulse variation patterns to use, how to define the sequence of different pulse trains, adjust a sequence of pulse trains, remove, add, or replace one or more pulse trains, or any other type of stimulation programming. As another example, the clinician may use programmer 20 to retrieve information from IMD 14 regarding the performance or integrity of IMD 14 or other components of system 10, such as lead 16 or a power source of IMD 14. In some examples, this information may be presented to the clinician as an alert if a system condition that may affect the efficacy of therapy is detected.

In some examples, a clinician may use clinician programmer 20 to create stimulation programs for electrical stimulation (generated and delivered by IMD 14) as therapy to treat bladder dysfunction of patient 12. In some examples, the clinician programmer 20 transmits the stimulation programs to IMD 14 for storage in a memory of IMD 14. Clinician programmer 20 may be configured to have additional functionality and/or control of IMD 14 than the patient programmer 22.

Patient programmer 22 facilitates interaction of patient 12 with one or more components of system 10. In some examples, patient 12 may interact with patient programmer 22 to control IMD 14 to deliver electrical stimulation, to manually abort the delivery of electrical stimulation by IMD 14, or to inhibit the delivery of electrical stimulation by IMD 14. Patient 12 may, for example, use a keypad or touch screen of programmer 22 to cause IMD 14 to deliver electrical stimulation, e.g., to activate one or more stimulation programs, or the like.

In some examples described herein, patient 12 may provide input to patient programmer 22 indicating that a bladder related event occurred. For example, in some examples, patient 12 may select a particular button of patient programmer 22 to indicate an occurrence of a bladder related event or provide input to a touch screen of patient programmer 22 indicating the occurrence of the bladder related event. The button can be a dedicated button that is designated to receive input from patient 12 indicating the bladder related event, or the button can be a multifunction button, such as a soft key that changes function depending upon the section of the user interface currently viewed by patient 12 (or another user). After receiving the input, patient programmer 22 may store the occurrence of the bladder related event, transmit the indication of the bladder related event to IMD 14, identify a timing of a plurality of bladder related events based on the bladder related event, or the like.

In other examples, one or more other components of therapy system 10 receives the patient input indicating the bladder related event. For example, in some examples, patient 12 interacts with IMD 14 to provide the input. As an example, IMD 14 can include a motion sensor integrated into or on a housing of IMD 14, where the motion sensor is configured to generate a signal that is indicative of patient 12 tapping IMD 14 through the skin. The number, rate, or pattern of taps may be associated with the different types of input, such as input indicating a bladder related event occurred, input indicating an intent to void, or the like. Patient 12 may provide the input by tapping IMD 14 and processing circuitry of IMD 14 may identify the tapping of IMD 14 by patient 12 to determine when patient input is received and to identify a timing of a plurality of bladder related events based upon receiving the patient input.

In some examples, it may be desirable to balance the repetitiveness of the therapy with muscle recovery times in order to help prevent muscle fatigue from the stimulation. In some examples, IMD 14 delivers multiple sessions of electrical stimulation daily or over another period of time, multiple cycles of electrical stimulation per session. During each stimulation session, IMD 14 may generate and deliver stimulation according to predetermined therapy programs. In some examples, patient 12 may determine when the delivery of electrical stimulation may be convenient, e.g., not disruptive, not embarrassing, or the like, for patient 12 and may provide input to patient programmer 22 to define the schedule of electrical stimulation delivery to accommodate these times, or provide input to patient programmer 22 that initiates the electrical stimulation delivery accordingly.

Patient programmer 22 may also receive other input from the patient regarding therapy. For example, patient programmer 22 may receive input from the patient that indicates the current therapy is acceptable (e.g., a “like” button) and/or receive input from the patient that indicates the current therapy is not acceptable (e.g., a “dislike” button). In response to receiving input that the therapy is acceptable, programmer 22, programmer 20, and/or IMD 14 may identify the pulse train deliverable at that time as acceptable to the patient. In response to receiving input that the therapy is not acceptable, programmer 22, programmer 20, and/or IMD 14 may identify the pulse train deliverable at that time as unacceptable to the patient. Programmer 22, programmer 20, and/or IMD 14 may remove any pulse trains identified as unacceptable. In some examples, programmer 22 or another device may replace a removed pulse train from a sequence with a new pulse train that has another different unique parameter variation pattern.

IMD 14, clinician programmer 20, and patient programmer 22 may communicate via wireless communication using any techniques known in the art. Examples of communication techniques may include, for example, low frequency or radiofrequency (RF) telemetry, or Bluetooth. Other techniques are also contemplated. In some examples, clinician programmer 20 and/or patient programmer 22 may include a programming head that may be placed proximate to the patient's body near the IMD 14 implant site in order to improve the quality or security of communication between IMD 14 and clinician programmer 20 and/or patient programmer 22.

As described herein, IMD 14 may be configured to deliver electrical stimulation therapy using a sequence of different trains of pulses. The processing circuitry that is configured to control stimulation may be included within or distributed between one or more of IMD 14, programmer 20, programmer 22, or other device. In one example, IMD 14 may control stimulation circuitry of IMD 14 to deliver a sequence of a plurality of trains of electrical stimulation pulses. Each train of the plurality of train of pulses may include respective pulses at least partially defined by a unique parameter variation pattern. The unique parameter variation pattern may specify how the values of one or more parameters that define the pulses of the train are varied within the train of pulses. The unique parameter variation pattern of each train may be selected from or be a part of a plurality of parameter variation patterns. IMD 14 may also control the stimulation circuitry to repeatedly deliver the sequence of the plurality of trains of electrical stimulation pulses.

In some examples, at least one parameter variation pattern of the plurality of parameter variation patterns defines a varied pulse width of at least some pulses of a train of electrical stimulation pulses. In other words, a varied pulse width would result in a parameter variation pattern in which at least some of the pulses within a train have different pulse widths. In some examples, at least one parameter variation pattern of the plurality of parameter variation patterns defines a varied inter-pulse interval between at least some pulses of a train of electrical stimulation pulses. In other words, a varied inter-pulse interval would result in a parameter variation pattern in which at least some of the inter-pulse intervals, or time periods between adjacent pulses, are different during the train of pulses. In some examples, at least one parameter variation pattern of the plurality of parameter variation patterns defines a varied frequency of at least some pulses of a train of electrical stimulation pulses. In other words, a varied frequency would result in a parameter variation pattern in which the pulse frequency of at least some pulses is different during the pulse train. In some examples, at least one parameter variation pattern of the plurality of parameter variation patterns defines a varied electrode combination of at least some pulses of a train of electrical stimulation pulses. In this manner, the varied electrode combination results in at least some of the pulses of a train of pulses being delivered from an electrode combination different than other pulses of the same train of pulses. The electrode combinations described herein may be unipolar or monopolar (e.g., an anode relatively far from a cathode) or multipolar (e.g., one or more cathodes relatively close to one or more anodes). Multipolar electrode combinations would include bipolar electrode combinations as one example.

The above examples of parameter variation patterns may result in all pulses of the same train of pulses having a different value (e.g., no pulse having the same value) for the specified parameter. In other examples, the parameter variation pattern may result in one or more pulses having a first value for the parameter and one or more other pulses have a second value for the parameter different than the first parameter. These variations may thus result in two or more different values for the same parameter used for the pulses within a single pulse train. Pulses of these two or more different values may, in some examples, be interleaved with one another to create the train of pulses. Although two different values for one parameter are described, three or more different values for the same parameter may be used within one parameter variation pattern in some examples. In addition, some parameter variation patterns for a single pulse train may include value variations for two or more parameters. As one example, two pulses within a single pulse train may have different values for a first parameter, and two pulses within the single pulse train have different values for a second parameter different than the first parameter. These two pulse from each example may be the same or different pulses such as some pulses within the pulse train may have different values for the two or more parameters or each pulse only varies by only one parameter values such that each pulse only deviates from a standard parameter set by one of the parameter values.

According to these examples, a plurality of parameter variation patterns can include a first unique parameter variation pattern defining a first parameter that is varied and a second unique parameter variation pattern defining a second parameter that is varied, where the first parameter being different than the second parameter. The sequence can include a first train of the plurality of trains of electrical stimulation pulses including pulses at least partially defined by the first unique parameter variation pattern and a second train of the plurality of trains of electrical stimulation pulses comprising pulses at least partially defined by the second unique parameter variation pattern. Of course, in some examples, three or more unique parameter variation patterns may be present for the multiple trains of any sequence. IMD 14 may be configured to repeat this sequence over a period of time. In some examples, IMD 14 may automatically adjust one or more trains, and the corresponding unique parameter variation pattern of the one or more trains, within the sequence. Alternatively, IMD 14, programmers 20 or 22, or any other controlling device may adjust the one or more trains within the sequence in response to feedback from a user (e.g., the patient or clinician) or a specific request to change one or more trains within the sequence.

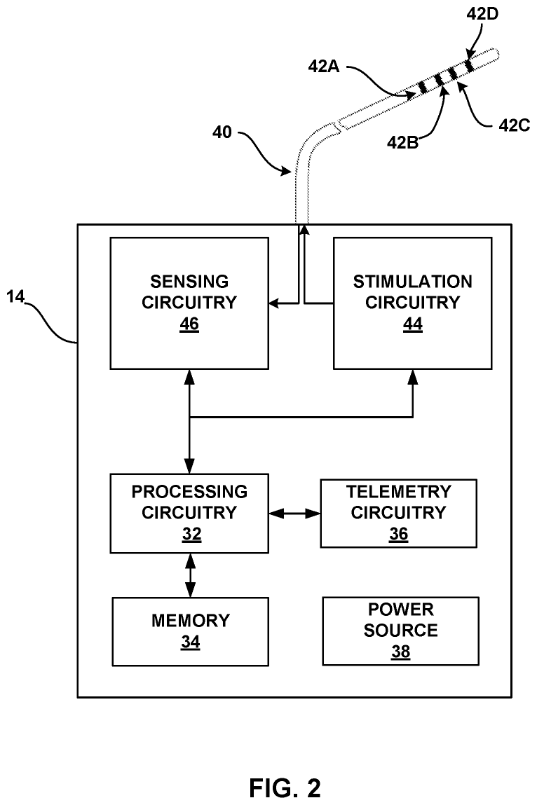

FIG. 2 is a conceptual block diagram illustrating an example of an IMD 14 configured to deliver therapy to a patient. As shown in FIG. 2, a stimulation circuitry 44 (e.g., electrical circuitry that may include a stimulation generator) of IMD 14 may generate electrical stimulation according to a plurality of electrical stimulation parameter sets or therapy programs. IMD 14 may deliver therapy to one or more nerves of patient 12 via one or more electrodes 42A-D (collectively, “electrodes 42”) positioned along lead 40. In some examples, IMD 14 may further include electrical sensing circuitry 46 for sensing a signal generated by one or more of nerves or one or more muscles in response to the electrical stimulation. The same or different electrodes may be used to generate stimulation and detect the response signal. In some examples, different sets of electrodes may be used to deliver stimulation and sense a physiological response to the delivered stimulation. IMD 14 may further include processing circuitry 32 that controls the operations of IMD 14 with the aid of instructions associated with program information that is stored in memory 34. IMD 14 may communicate with an external clinician programmer 20, external patient programmer 22, or another external device via telemetry circuitry 36.

Processing circuitry 32 may include one or more processors, such as microprocessors, digital signal processors (DSPs), application specific integrated circuits (ASICs), field programmable gate arrays (FPGAs), or any other equivalent integrated or discrete logic circuitry, as well as any combinations of such components. Memory 34 may include memory, such as random access memory (RAM), read only memory (ROM), programmable read only memory (PROM), erasable programmable read only memory (EPROM), electronically erasable programmable read only memory (EEPROM), flash memory, comprising executable instructions for causing the one or more processors to perform the actions attributed to them. Further, memory 34 may be implanted entirely in hardware, software, or a combination thereof.

In some examples, processing circuitry 32 of IMD 14 is configured to deliver the sequence of pulse trains according to a predetermined schedule or in response to one or more trigger events, such as identification of an upcoming bladder related event, a sensed parameter (e.g., bladder pressure or volume) exceeding a threshold, patient request for therapy, or any other type of feedback. In some examples, a sensed parameter may be sensed from nerves, muscles, or other tissue different from the bladder.

In some examples, IMD 14 may possess one or more electrodes 42 coupled to IMD 14 via one or more leads 40. Electrodes 42 may be configured to deliver electrical stimulation according to a plurality of electrical stimulation parameter sets or therapy programs generated by processing 32 and stored in memory 34. Electrodes 42 may operate as a cathode or an anode. Electrodes 42 may be any type of electrode, such as a ring electrode, segmented electrode, paddle electrode, cuff electrode, needle electrode, or plate electrode. Electrodes 42 are typically implanted electrodes disposed internal of the patient. However, one or more of electrodes 42 may be external to the patient in some examples. In some examples, Electrodes 42 may be implanted adjacent to or even coupled to one or more of a patient's nerve fibers. In some examples, electrode 42 may be implanted adjacent to or even coupled to (e.g., implanted at least partially within) a tissue or muscle fiber. In some examples, more than one electrode may be coupled to the same nerve. In some examples, electrodes 42 may be coupled to a bundle of nerves or muscle fibers. In this manner, one or more electrodes of electrode 42 may be disposed adjacent to, around (e.g., a cuff electrode), or even within a nerve or muscle. Although four electrodes 42 are shown in the example of FIG. 2, fewer than four or more than four electrodes may be carried by lead 40, or multiple leads, in other examples. In some examples, some of electrodes 42 may be positioned to deliver stimulation while other electrodes 42 may be positioned to detect physiological responses from delivered stimulation. In other examples, the same electrodes of electrodes 42 may be configured to deliver stimulation and sense electrical signals. In other examples, the same electrodes that delivered stimulation may be used to detect nerve and/or muscle responses evoked from the delivered stimulation in order to titrate stimulation.

Each electrical stimulation parameter set or therapy program generated by processing circuitry 32 may define an electrical stimulation signal deliverable to a patient. In some examples, the electrical stimulation parameter set or therapy program may include values for voltage or current amplitude, pulse frequency, pulse width, pulse polarity, and/or electrode combination. In some examples, the electrical stimulation parameter set may also define the recharge phase of a pulse, such as amplitude and/or pulse width for active recharge phases or whether the recharge phase is passive. These values of the voltage or current amplitude over time for each pulse may define the waveshape of each pulse or signal (e.g., rectangular, sinusoidal, Gaussian, sawtooth, rising, falling, etc.). In addition, each stimulation parameter set may define a burst of pulses and a frequency of the burst of pulses instead of a continuous pulse train. Different stimulation parameter sets or therapy programs may vary by a different value for at least one of the stimulation parameters. In some examples, the electrical stimulation parameter set or therapy program may include the number of pulses or signals or the duration for which pulses are to be delivered. The electrode combination may define which electrodes are used to deliver stimulation signals and the polarity (cathode or anode) of each electrode. In some examples, the electrical stimulation parameter set may define electrical stimulation at, or below, a perception threshold (e.g., the level at which the stimulation is perceived by the patient), a motor threshold (e.g., the level at which a muscle response is induced), and/or an activation threshold (e.g., the level at which the nerve is depolarized to activate the nerve) of the patient.

In one example of the system described herein, processing circuitry 32 may select an electrical stimulation parameter set (e.g., values for respective parameters) or therapy program based on parameter variation patterns that will be part of the sequence of pulse trains. For example, IMD 14 may deliver electrical stimulation therapy to the patient via electrodes 42A and 42B for one pulse train and electrodes 42B and 42 for another pulse train of the sequence. In other examples, one or more additional or alternative electrodes 42 may be used to deliver the electrical stimulation therapy to the patient. In some examples, electrical sensing circuitry 46 of IMD 14 may obtain a signal representative of an electrical response sensed from the patient in response to the electrical stimulation delivered to the patient according to the respective electrical stimulation parameter set or therapy program.

The electrical response obtained from the patient may be a measured voltage or a measured current from nerves and/or muscles and sensed by electrodes 42. In one example, electrode 42B senses a measured voltage response of a nerve fiber in response to the electrical stimulation delivered to the same nerve fiber according to the respective electrical stimulation parameter set. In another example, electrode 42B senses an electromyogram (EMG) signal of the patient. In another example, electrode 42B senses a nerve recording of an action potential of one or more nerves of the patient. In another example, electrode 42B senses a composite bioelectrical signal corresponding to an activity of nerve and muscle fibers of the patient. In another example, electrode 42B senses a respective movement signal representative of a motion of a portion of the patient in response to the electrical stimulation. In this manner, one or more electrodes 42 may be configured to deliver electrical stimulation signals and/or sense evoked responses from tissue. In some examples, the measured response to stimulation may be used by IMD 14 or another device to adjust the sequence to remove, replace, or add pulse trains to the sequence, or adjust when the sequence is delivered to the patient (e.g., reduce or increase the duty cycle of the sequence).

Electrical sensing circuitry 46 may receive respective sensed signals from one or more electrodes, such as electrode 42B, evoked from the electrical stimulation delivered according to the electrical stimulation parameter set. In some examples, electrical sensing circuitry 46 may perform signal processing of each received signal to remove noise or other unwanted frequencies or artifacts (e.g. stimulation, motion, cardiac etc.). Electrical sensing circuitry 46 may also convert the analog signal to a digital signal and/or provide other signal processing functionality. Processing circuitry 32 may operate in conjunction with electrical sensing circuitry 46 to evaluate or analyze the received signal for one or more characteristics, such as one or more signal peaks, peak amplitudes, number of peaks, areas under peaks, peak widths, time between peaks, ratios of peak amplitudes, widths, and/or areas, peak latency, signal valleys, valley amplitudes, number of valleys, areas above valleys, valley widths, time between valleys, ratios of valley amplitudes, widths, and/or areas, valley latency, root-mean-square signal value, signal skew, kurtosis, frequency and/or spectral content of the signal(s), or any other suitable signal feature. Using one or more of these characteristics of the sensed signal, processing 32 may determine whether or not the respective parameter set defined effective stimulation or if a different parameter set may be more effective. Once processing circuitry 32 determines that a parameter set defined stimulation that evoked a desired response from the patient, processing circuitry 32 may update the sequence of pulse trains to include one or more pulse trains that are defined by the parameter set. In some examples, processing circuitry 32 may adjust one or more pulses in one or more pulse trains to use the new parameter value identified as being more effective instead of a previous parameter value. In some examples, processing circuitry 32 may remove pulse trains using pulses of the old parameter value and/or add pulse trains using pulses defined by the new parameter value identified to be more effective for therapy.

Additionally, or alternatively, electrical sensing circuitry 46 may be configured to determine a pressure or a volume of a bladder, a physical activity, a time of day, an amount of fluid intake, and/or an amount of caffeine consumed by the patient. This data may be received via sensors from the IMD, programmer, or other device monitoring the patient or via patient input. As one example, electrical sensing circuitry may receive respective sensed signals from one or more electrodes 42, which may be analyzed by processing circuitry 32 to determine the pressure or the volume of the bladder of the patient. In turn, processing circuitry 32 may start and/or stop delivery of therapy using the sequence of pulse trains based on the pressure and/or the volume of the bladder. In some examples, processing circuitry 32 may adjust a duty cycle of the sequence of pulse trains based on the need for therapy, such as increasing the duty cycle in response to determining that the bladder has increased in volume (or exceeded one or more volume or pressure thresholds) and decreasing the duty cycle in response to determining that the bladder has been emptied (e.g., a voiding event occurred).

The architecture of IMD 14 illustrated in FIG. 2 is shown for exemplary purposes only. The techniques as set forth in this disclosure may be implemented in the example IMD 14 of FIG. 2, as well as other types of IMDs not described specifically herein. For example, processing circuitry 32 may be located within IMD 14, or within an external programming device used to configure or control IMD 14 remotely. Further, electrical sensing circuitry 46 may be located within IMD 14, and/or within an external programming device that senses nerve response to electrical stimulation via one or more external electrodes or senses another physiological event of the patient (e.g., a pressure or a volume of a bladder, a physical activity, a time of day, an amount of fluid intake, and/or an amount of caffeine consumed by the patient). In other examples, IMD 14 may include or be in direct communication with other sensors, such as one or more accelerometers, pressure sensors, flow sensors, wetness sensors, or any other sensors that may provide information indicative of bladder or bowel related events of patient 12.

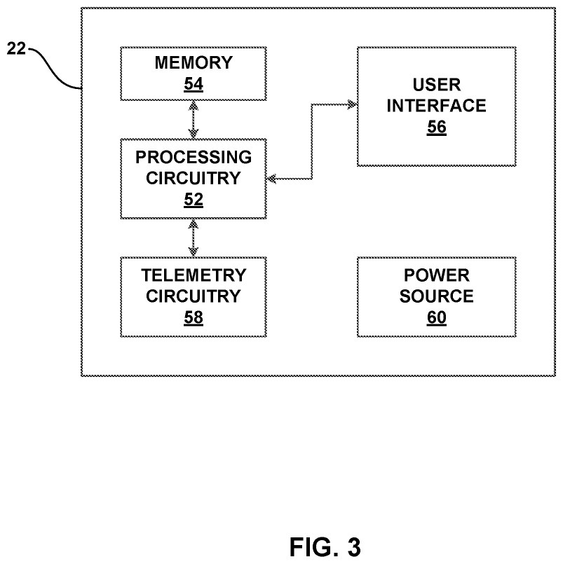

FIG. 3 is a block diagram illustrating an example patient programmer 22. While patient programmer 22 may generally be described herein as a hand-held computing device, in other examples, patient programmer 22 may be a notebook computer, tablet computer, a cell phone, smart phone, or a workstation, or a remote/key fob for example. As illustrated in FIG. 3, patient programmer 22 may include a processing circuitry 52, memory 54, telemetry circuitry 58, user interface 56, and power source 60. Memory 54 may store program instructions that, when executed by processing circuitry 52, cause processing circuitry 52 and patient programmer 22 to provide the functionality ascribed to patient programmer 22 throughout this disclosure. Clinician programmer 20 may include similar components to patient programmer 22.

In some examples, memory 54 may further include program information, e.g., stimulation programs similar to those stored in memory 34 of IMD 14. In some examples, the stimulation programs stored in memory 54 may be downloaded into memory 34 of IMD 14. Memory 54 may include any volatile, non-volatile, fixed, removable, magnetic, optical, or electrical media, such as RAM, ROM, CD-ROM, hard disk, removable magnetic disk, memory cards or sticks, NVRAM, EEPROM, flash memory, and the like. Processing circuitry 52 can take the form one or more microprocessors, DSPs, ASICs, FPGAs, programmable logic circuitry, or the like, and the functions attributed to processing circuitry 52 herein may be embodied as hardware, firmware, software or any combination thereof.

User interface 56 is configured to receive input from a user and may include, for example, a button or keypad, lights, a speaker for voice commands, a display, such as a LCD, LED, or CRT. In some examples, the display may include a touch sensitive screen. In some examples, processing circuitry 52 may receive patient input, e.g., patient input indicating a bladder related event, via user interface 56. The input may be, for example, in the form of pressing a button on a keypad or selecting an icon from a touch screen. This patient input may be received by an IMD, programmer, smartphone, or any other device. In response to receiving the input, processing circuitry 52 may, for example, control telemetry circuitry 58 to deliver a signal that indicates receipt of the input to IMD 14, clinician programmer 20, or another device. For clinician programmer 20, a user interface similar to user interface 56 may represent selectable icons, drop down menus, text entry fields, numerical entry fields, or any other input mechanisms for the clinician to select parameter values that define pulses within each train of pulses, select pulse trains for a sequence, select the timing of each pulse train within each sequence, select the duty cycle for the sequence as it is repeated over time, or any other criteria that defines the delivery of a sequence of pulse trains as described herein.

Processing circuitry 52 may also be configured to present information, e.g., information related to one or more sessions of electrical stimulation, electrical stimulation parameters, schedules of delivery of electrical stimulation, initiation of a particular stimulation session, available parameter variation patterns, parameter variation patterns for pulse trains, pulse trains selected for a sequence, sequence parameters, and the like, to patient 12 or another user (e.g., a patient caretaker) via user interface 56. Although not shown, patient programmer 22 may additionally or alternatively include a data or network interface to another computing device, to facilitate communication with another device, e.g., IMD 14, and presentation of information relating to electrical stimulation via the other device.

Telemetry circuitry 58 supports wireless communication between IMD 14 and patient programmer 22 under the control of processing circuitry 52. Telemetry circuitry 58 may also be configured to communicate with another computing device, such as clinician programmer 20, via wireless communication techniques, or direct communication through a wired connection. Telemetry circuitry 58 may be substantially similar to telemetry circuitry 36 described above, providing wireless communication via an RF or proximal inductive medium. In some examples, telemetry circuitry 58 may include an antenna, which may take on a variety of forms, such as an internal or external antenna. An external antenna that is coupled to patient programmer 22 may correspond to a programming head that may be placed over IMD 14.

Examples of local wireless communication techniques that may be employed to facilitate communication between patient programmer 22 and another computing device include RF communication according to the 802.11 or Bluetooth® specification sets, infrared communication, e.g., according to the IrDA standard, or other standard or proprietary telemetry protocols. In this manner, other external devices may be capable of communicating with patient programmer 22 without needing to establish a secure wireless connection.

IMD 14, patient programmer 22, and/or clinician programmer 20 may control the delivery of electrical stimulation according to one or more stimulation programs that define the pulse trains within a sequence of and the sequence. In some examples, the one or more stimulation programs may also define how the sequence is delivered, such as the frequency of the sequence repeating, duty cycle of the sequence, closed-loop feedback parameters for automatic adjustment of the sequence and/or pulse trains within the sequence, etc. In some examples in which patient programmer 22 controls the stimulation, patient programmer 22 may transmit stimulation programs (e.g., the actual parameter values or an indication of the stimulation program) for implementation by IMD 14 to IMD 14 via telemetry circuitry 58. In some examples, a user (e.g., patient 12 or a clinician) may select one or more stimulation programs from a list provided via a display of user interface 56. Alternatively, patient programmer 22 may transmit a signal to IMD 14 indicating that IMD 14 should execute locally stored programs or therapy schedules. In such a manner, control over the electrical stimulation may be distributed between IMD 14 and patient programmer 22, or may reside in either one alone.

Power source 60 is configured to deliver operating power to the components of patient programmer 22. Power source 60 may include a battery and a power generation circuit to produce the operating power. In some examples, the battery may be rechargeable to allow extended operation. Recharging may be accomplished by electrically coupling power source 60 to a cradle or plug that is connected to an alternating current (AC) outlet. Additionally, or alternatively, recharging may be accomplished through proximal inductive interaction between an external charger and an inductive charging coil within patient programmer 22. In other examples, traditional batteries (e.g., nickel cadmium or lithium ion batteries) may be used. In addition, patient programmer 22 may be directly coupled to an alternating current outlet to power patient programmer 22. Power source 60 may include circuitry to monitor power remaining within a battery. In this manner, user interface 56 may provide a current battery level indicator or low battery level indicator when the battery needs to be replaced or recharged. In some cases, power source 60 may be capable of estimating the remaining time of operation using the current battery.

In some examples, clinician programmer 20 includes components similar to those of patient programmer 22 shown in FIG. 3. However, other configurations of clinician programmer 20 are contemplated.

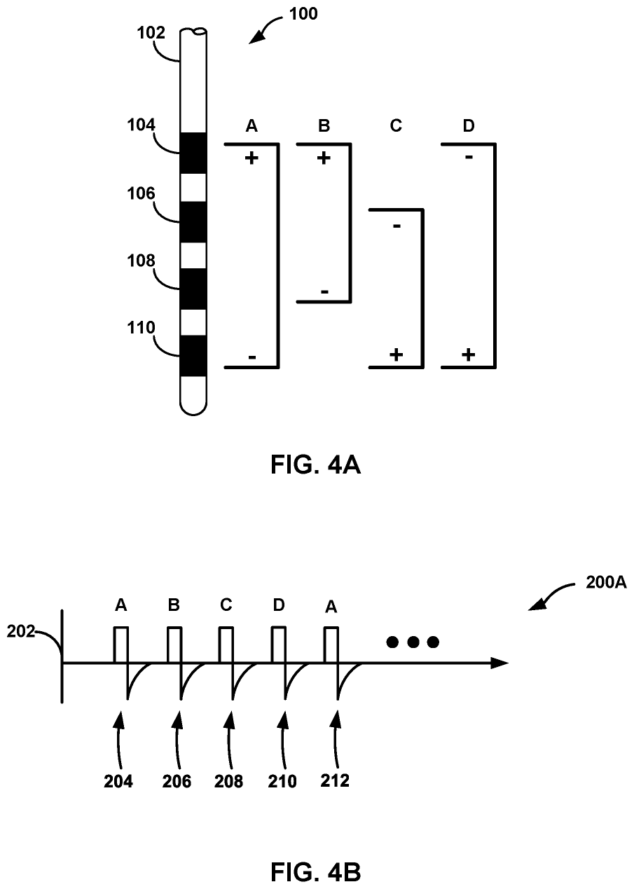

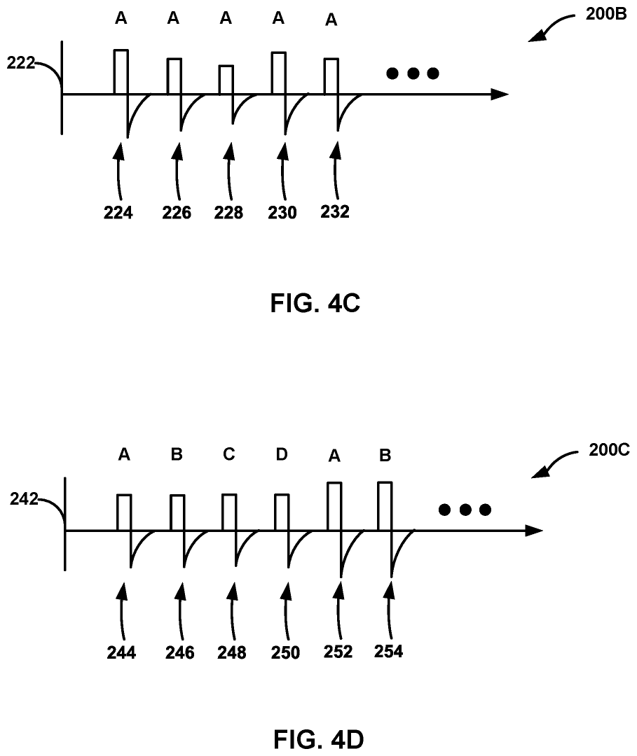

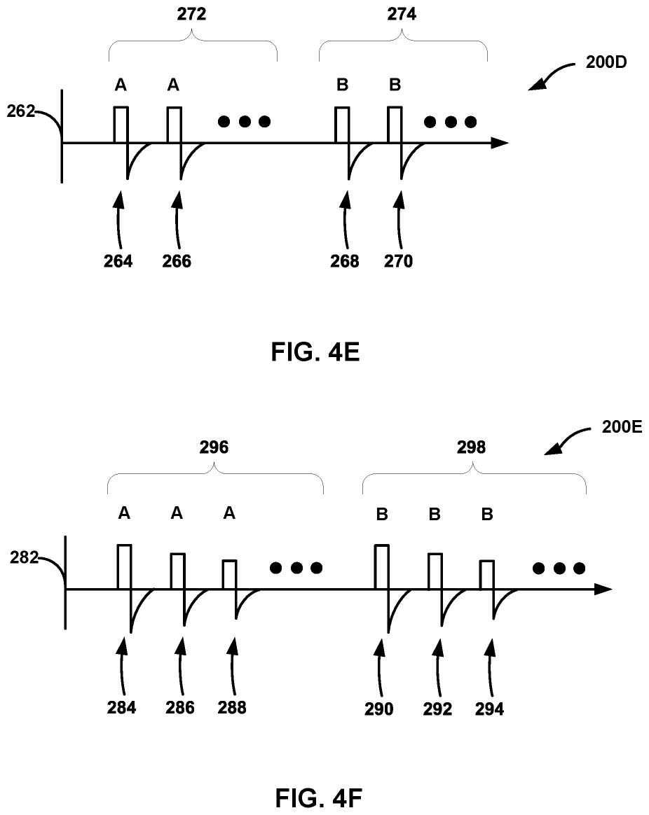

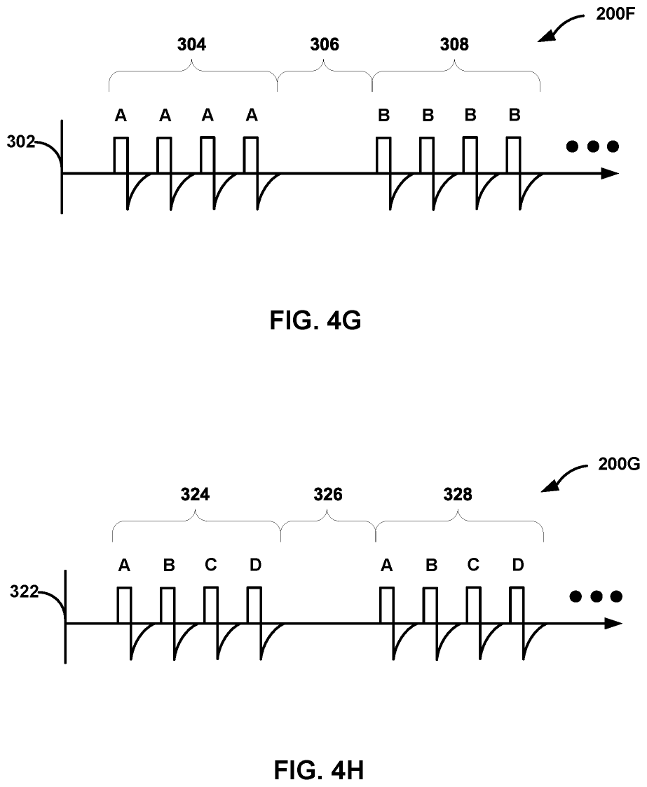

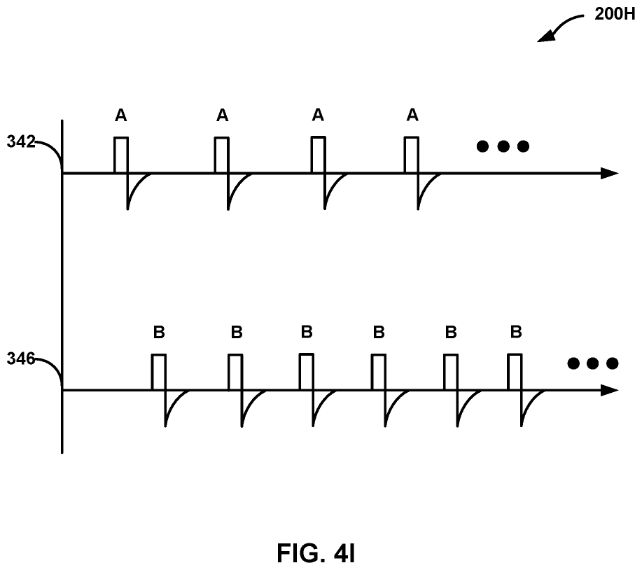

FIG. 4A is a conceptual diagram of an example lead and electrode combinations. As shown in FIG. 4A, lead 102 is a medical lead that includes four cylindrical electrodes at different axial locations along the length of lead 102. Lead 102 is an example of leads 16 and 14, but other leads may have one, two, three, five, six, or more cylindrical electrodes. In some examples, one or more electrodes may be segmented electrodes that are electrodes located only partially around the perimeter of lead 102. For example, lead 102 may include one or more axial locations along the lead that include one, two, three, four, or more electrodes at the same axial location but at different respective circumferential locations around the housing of lead 102. Each of these electrodes may be independently selected to be a cathode or anode to deliver a stimulation pulse and/or sense signals from the patient. In some examples, the housing of IMD 14, for example, may carry one or more electrodes that can sink or source current from or to one or more electrodes of lead 102.

In the example of FIG. 4A, lead 102 includes four electrodes 104, 106, 108, and 110. These four electrodes may be used as cathodes or anodes in different electrode configurations. Configuration A indicates that electrode 104 is an anode and electrode 110 is a cathode. Configuration B indicates that electrode 104 is an anode and electrode 108 is a cathode. Configuration C indicates that electrode 110 is an anode and electrode 106 is a cathode. Configuration D indicates that electrode 110 is an anode and electrode 104 is a cathode. These are just example electrode configurations A, B, C, and D that are possible using a four electrode lead 102 and will be described below to illustrate possible parameter variation patterns for pulse trains of a sequence of pulse trains for electrical stimulation. However, other electrode configurations are possible, such as electrode 106 being an anode and electrode 108 being a cathode, electrode configurations that include three or four electrodes (e.g., electrode 106 is a cathode and electrodes 104 and 108 are anodes). In some examples, electrode configurations may be provided between two or more electrodes distributed across two or more leads. In some examples, electrode configurations may be provided between one or more electrode as the cathodes and the IMD 14 as the anode. Therefore, the electrode configurations A, B, C, and D of FIG. 4A are merely examples of some different electrode configurations that are possible for parameter variation patterns.

FIGS. 4B, 4C, 4D, 4E, 4F, 4G, 4H, and 4I are timing diagrams for pulse trains with pulses that are varied according to example parameter variation patterns. As discussed above, lead 102 and electrode configurations A, B, C, and D will be discussed with respect to the example parameter variation patterns for purposes of illustration, but any electrode combinations possible with available electrodes are also contemplated. Each parameter variation pattern may include at least one value for at least one parameter that is different between at least two pulses of a pulse train. The parameters that may be varied include electrode combination, current or voltage amplitude, pulse width, pulse frequency, pulse polarity, interphase interval, interburst frequency, intraburst frequency (e.g., the frequency of pulses within a burst of pulses), a pulse train schedule, or any other parameter. In some examples, pulses may be configured to have a pulse width from approximately 1 microseconds (μs) to approximately 450 ids, a pulse frequency from approximately 1 Hz to approximately 5 kHz, an interphase interval (e.g., the time between a first phase and an active or passive phase of a pulse) from approximately 10 ids to approximately 250 ids (or set as a percentage of pulse width), a interburst frequency from approximately 1 Hz to approximately 5 kHz, an intraburst pulse frequency from approximately 1 Hz to approximately 5 kHz, and a scheduling frequency for a train from approximately 1 second to several days. In some examples, the pulse frequency may be less than 1,200 Hz, such as from about 1 Hz to about 1,200 Hz. Other parameters may include inter-pulse interval, which can alternatively be defined as a function of the pulse frequency and pulse width.

The system, such as IMD 14, programmers 20 or 22, alone or in combination, may control stimulation circuitry of IMD 14 to deliver a sequence of a plurality of trains of electrical stimulation pulses. Each train of the plurality of trains of electrical stimulation pulses may include respective pulses at least partially defined by a unique parameter variation pattern. Each parameter variation pattern, such as the parameter variation patterns of example FIGS. 4B-4I, includes pulses defined by a parameter set in which the value for at least one parameter is different for one or more pulses of the pulse train compared to one or more other pulses of the pulse train. Therefore, each parameter variation pattern defines at least one varied parameter, such as one or more of a varied pulse width, varied inter-pulse interval, varied frequency, and varied electrode combination.

As shown in the example of FIG. 4B, parameter variation pattern 200A includes a varied electrode combination between different pulses of pulse train 202. Pulse train 202 includes pulses 204, 206, 208, 210, 212, and other pulses that may continue as part of the train. The electrode combination variation is varied between the four electrode combinations A, B, C, and D. Pulse 204 is delivered via electrode combination A, pulse 206 is delivered via electrode combination B, pulse 208 is delivered via electrode combination C, pulse 210 is delivered via electrode combination D, and pulse 212 is delivered via electrode combination A once again. In this manner, parameter variation pattern 200A changes the electrode combination for the next pulse in pulse train 202 as IMD 14 cycles between electrode combinations A, B, C, and D. The other stimulation parameters that define the pulses of pulse train 202 may be the same, such that only the electrode combination is varied. Alternatively, one or more other parameters may also be varied for one or more of the pulses. In some examples, a parameter such as amplitude may be adjusted for respective electrode combinations in order to achieve desired stimulation intensities from each electrode combination.

As shown in the example of FIG. 4C, parameter variation pattern 200B includes a varied electrode combination between different pulses of pulse train 222. Pulse train 222 includes pulses 224, 226, 228, 230, 232, and other pulses that may continue as part of the train. Each pulse in pulse train 222 is delivered via the same electrode combination A. The electrode combination variation is an amplitude variation that changes between three different amplitudes. Pulse 224 is delivered at a first amplitude, pulse 226 is delivered at a second amplitude lower than the first amplitude, and pulse 228 is delivered with a third amplitude lower than the second amplitude. Then, that amplitude variation is cycled again starting with pulse 230 at the first amplitude and pulse 232 at the second amplitude, etc. The amplitude variation in parameter variation pattern 200B specifically is a repeating step-down amplitude, but other examples may have repeating step-up amplitude variation, step-down and step-up repeating cycles (e.g., an increasing and then decreasing “wave” of amplitude values), random amplitude variation, or any other type of amplitude change between one or more pulses of pulse train 222. The other stimulation parameters that define the pulses of pulse train 202 may be the same, such that only the amplitude is varied. Alternatively, one or more other parameters may also be varied for one or more of the pulses. For example, a parameter such as pulse width may be adjusted in conjunction with the amplitude such that the overall intensity is maintained for each pulse (e.g., a pulse with an increased amplitude may have a decreased pulse width).

As shown in the example of FIG. 4D, parameter variation pattern 200C includes a varied electrode combination between different pulses of pulse train 242. Pulse train 242 includes pulses 244, 246, 248, 250, 252, and other pulses that may continue as part of the train. The electrode combination variation is varied between the four electrode combinations A, B, C, and D. Pulse 244 is delivered via electrode combination A, pulse 246 is delivered via electrode combination B, pulse 248 is delivered via electrode combination C, pulse 250 is delivered via electrode combination D, and then pulse 252 is delivered via electrode combination A once again followed by pulse 254 via electrode combination B. In this manner, parameter variation pattern 200C changes the electrode combination for the next pulses in pulse train 242 as IMD 14 cycles between electrode combinations A, B, C, and D. In addition, parameter variation pattern 200C varies the amplitude of the pulses between different cycles through the different electrode combinations. Pulses 252 and 254 have an amplitude greater than pulses 244, 246, 248, and 250. Parameter variation pattern 200C may only alternate between two amplitudes for the repeating cycles through the electrode combinations or continue to use third different amplitude for the third cycle (or additional different amplitudes) of electrode combinations. Although parameter variation pattern 200C shows the amplitude and electrode combination changing in cycles, the changes may have other changing patterns such as repeating values between adjacent pulses and/or random changes in the amplitude and/or electrode combination between adjacent pulses. The other stimulation parameters that define the pulses of pulse train 242 may be the same, such that only the electrode combination and amplitude is varied within parameter variation pattern 200C. Alternatively, other parameters may also be varied for one or more of the pulses. In some examples, a parameter such as pulse width may be adjusted for respective electrode combinations in order to achieve desired stimulation intensities from each electrode combination.

As shown in the example of FIG. 4E, parameter variation pattern 200D includes a varied electrode combination between different bursts of pulse within train 262. This variation pattern may be referred to as contact scheduling, or electrode combination scheduling, such that pulses may be delivered at each electrode combination for the same or different periods of time. Pulse train 262 includes burst 272 of pulses 264, 266, and possibly others not shown, as well as at least one more burst 274 of pulses 268, 270, and possibly others not shown. The pulses of burst 272 may have the same stimulation parameter values, such as the same electrode combination, and amplitude. The pulses of burst 274 may have the same stimulation parameter values, but the pulses of burst 274 have a different electrode combination B than the pulses of burst 272 (electrode combination A). Therefore, parameter variation pattern 200D varies the electrode combination between different bursts 272 and 274 of pulses. Only two bursts 272 and 274 are shown, but three or more different bursts of pulses with the same or different electrode combinations may be provided in some examples. In addition, each burst of pulses may have a different number of pulses and/or different duration of time where pulses are delivered via the same electrode combination in order to adjust the time pulse are delivered to a certain tissue region. Thus, the different bursts of pulses within pulse train 262 may have the same number of pulses or different numbers of pulses. The inter-burst duration between each burst of pulses within pulse train 262 may be constant or changed in various examples.

The example parameter variation pattern 200E of FIG. 4F may be similar to the parameter variation pattern 200D of FIG. 4E. However, as shown in the example of FIG. 4F, parameter variation pattern 200E includes a varied electrode combination between different bursts of pulse within train 282 and a varied amplitude within each burst 296 and 298 within pulse train 282. The duration of time and/or pulses for each burst may also be varied to change how long stimulation is delivered at each tissue region. Pulse train 282 includes burst 296 of pulses 284, 286, 288, and possibly others not shown, as well as at least one more burst 298 of pulses 290, 292, 294, and possibly others not shown. The pulses of burst 296 have different amplitudes (as shown as the decreasing amplitude between pulses 284, 286, and 288). Burst 298 also shows the same amplitude variation between pulses 290, 292, and 294. In addition, the pulses of burst 296 may have a different electrode combination A than the pulses of burst 298 (electrode combination B). Therefore, parameter variation pattern 200E varies the electrode combination between different bursts 296 and 298 and the amplitudes of the pulses within each of bursts 296 and 298. Only two bursts 296 and 298 are shown, but three or more different bursts of pulses with the same or different electrode combinations and/or amplitudes may be provided in some examples. The inter-burst duration between each burst of pulses within pulse train 282 may be constant or changed in various examples. Although the variation of electrode combinations and amplitudes are provided in the example of FIG. 4F, other parameters may be varied in other examples.

FIG. 4G illustrates pulse train 302 which includes parameter variation pattern 200F that has a varied electrode combination and varied inter-burst duration between different bursts of pulses within train 302. Pulse train 302 includes burst 304 of pulses (four pulses are shown, but fewer or greater numbers of pulses may be used in other examples) and burst 308 of pulses (four pulses are shown, but fewer or greater numbers of pulses may be used in other examples). Bursts 304 and 308 may include the same number or different number of pulses. The pulses of burst 304 are delivered via electrode combination A, but the electrode combination is varied because burst 308 includes pulses delivered via electrode combination B. Pulse train 302 may include these bursts 304 and 308 repeated or include addition bursts of pulses delivered via different electrode combinations.

Inter-burst duration 306 is the amount of time between adjacent bursts 304 and 308. In some examples, inter-burst duration 306 is the same between all bursts of pulse train 302. In other examples, parameter variation pattern 200F may change this inter-burst duration 306 between subsequent bursts of pulses within pulse train 302. Therefore, parameter variation pattern 200F may, in some examples, vary both the electrode combination and inter-burst interval for pulse train 302. Only two bursts 304 and 308 are shown, but three or more different bursts of pulses with the same or different electrode combinations may be provided in some examples.

The example parameter variation pattern 200G of FIG. 4H may be similar to the parameter variation pattern 200F of FIG. 4G. However, as shown in the example of FIG. 4H, parameter variation pattern 200G includes a varied electrode combination within each burst of pulses 324 and 328. Pulse train 322 includes burst 324 of pulses as well as at least one more burst 328 of pulses, and possibly other bursts not shown. The pulses of burst 324 may be delivered at the same amplitude, but each pulse within the burst is delivered via a different one of electrode combinations A, B, C, and D. After inter-burst duration 326, burst 328 also includes pulses delivered at the same amplitude, but via different electrode combinations A, B, C, and D. Therefore, parameter variation pattern 200E varies the electrode combination within each burst of pulses. The amplitudes of the pulses within each of bursts 324 and 328 may be constant or varied, for example. Only two bursts 324 and 328 are shown, but three or more different bursts of pulses with the same or different electrode combinations and/or amplitudes may be provided in some examples. The inter-burst duration between each burst of pulses within pulse train 322 may be constant or changed in various examples.

FIG. 4I illustrates an example timing diagraph for parameter variation pattern 200H which incorporates variation of frequency and electrode combinations by delivering at least two pulse trains 342 and 346 by different electrode combinations A and B, respectively. For example, the pulses of pulse train 342 may be delivered via electrode combination A at a first pulse frequency, and the pulses of pulse train 346 may be delivered via a different electrode combination B. Since the pulse frequencies of pulse trains 342 and 346 are different, the pulses may need to be delivered via different electrode combinations in order to maintain the respective pulse frequencies. In other examples, IMD 14 may deliver the pulses of pulse train 342 and 346 such that if any pulses overlap, IMD 14 drops an overlapping pulse and continues with the next pulse that would be delivered. Delivering pulses to the different electrode combinations and different frequencies may enable IMD 14 to recruit different types of nerve fibers. In some examples, IMD 14 may also be able to recruit these different types of nerve fibers using lower amplitudes than if a pulse train from a single electrode combination and/or different frequency was delivered. The pulses of pulse trains 342 and 346 may have the same or different amplitude, same or different pulse width, or other similar or different parameter values.

IMD 14 may be configured to deliver stimulation with pulses that vary parameters according to any of the parameter variation patterns described herein, a combination of the parameter variation patterns, or other parameter variation pattern. IMD 14 may also deliver the stimulation using a sequence of multiple trains of pulses, where each train of pulses is defined by a unique, or different, parameter variation pattern. IMD 14 may be configured to separate consecutive trains of the plurality of trains of electrical stimulation pulses within the sequence by an inter-train delay period. In other words, the different pulse trains may be delivered back to back or with an inter-train delay period that separates the different pulse trains by a certain period of time. In some examples, the trains of a sequence may include the same number of pulses and/or continue for a same duration of time. In other examples, two or more trains of the sequence may have a different number of pulses and/or a different duration for which each train of pulses occurs. In this manner, the duration of time during which each respective train within a sequency is delivered may be different for different trains. In some example, this different duration of time for a train may result in a variation of pulse patterns between the trains.

In addition, IMD 14 may be configured to deliver the stimulation by repeating the sequence of trains of pulses over time. In some examples, IMD 14 may be configured to continuously deliver the sequence without delay between the repetitions of the sequence. In other examples, IMD 14 may be configured to separate consecutive deliveries of the sequence by an inter-sequence delay period. In other words, IMD 14 may deliver stimulation according to the sequence of pulse trains and then delay delivery of the sequence again for some period of time. In this manner, IMD 14 may turn the sequence on or off, or otherwise adjust a duty cycle of the sequence of pulse trains. Reducing the amount of time that stimulation is delivered may maintain patient response to stimulation over time and/or increase battery life over use of IMD 14 by consuming less power. In some examples, the repeatable sequence of pulse trains may include two or more pulse trains that each have a unique parameter variation pattern. In some examples, the sequence of the plurality of trains of electrical stimulation pulses may include at least four trains of electrical stimulation pulses, where each train of the four trains of electrical stimulation pulses includes respective pulses that are varied according to respective unique parameter variation patterns of the plurality of parameter variation patterns. In some examples, a sequence may repeat one or more pulse train such that not all trains within a sequence have a unique parameter variation patterns.

During stimulation, IMD 14 may provide one or more adjustments to one or more stimulation parameters that define the pulses within a pulse train and/or within the entire sequence of pulse trains. For example, in response to receiving user request to a change to amplitude, IMD 14 may make that requested change to amplitude to all pulses within the trains of the sequence. This type of parameter adjustment may be referred to as a global amplitude adjustment because it will apply to all pulses to be delivered to the patient. This global adjustment may be the same magnitude of amplitude change for all pulses or a scaled amplitude change to maintain a ratio of amplitude change across all of the subsequent pulses that may be at different amplitude values or may apply to only pulses above the new user defined maximum threshold.

In some examples, some or all of the pulses within a sequence may be configured to provide stimulation that is perceptible by the patient (e.g., supra-perception threshold), such as paresthesia to reduce the perception of pain. In other examples, some or all of the pulses, or some or all of the pulse trains within a sequence, may be configured to be sub-perception threshold pulses that may provide relief from stimulation. In any event, the pulses described herein may be configured to be at, below or above perception threshold or paresthesia threshold. In other examples, some or all of the pulses within a sequence and/or set of sequence patterns may be configured according to the signal threshold measured by the sensing electrode(s) on the stimulation lead. The measured signal threshold would define one or more stimulation parameters (e.g., amplitude, pulse frequency, etc.), and IMD 14 may configured the rest of the pattern(s) around these parameters.

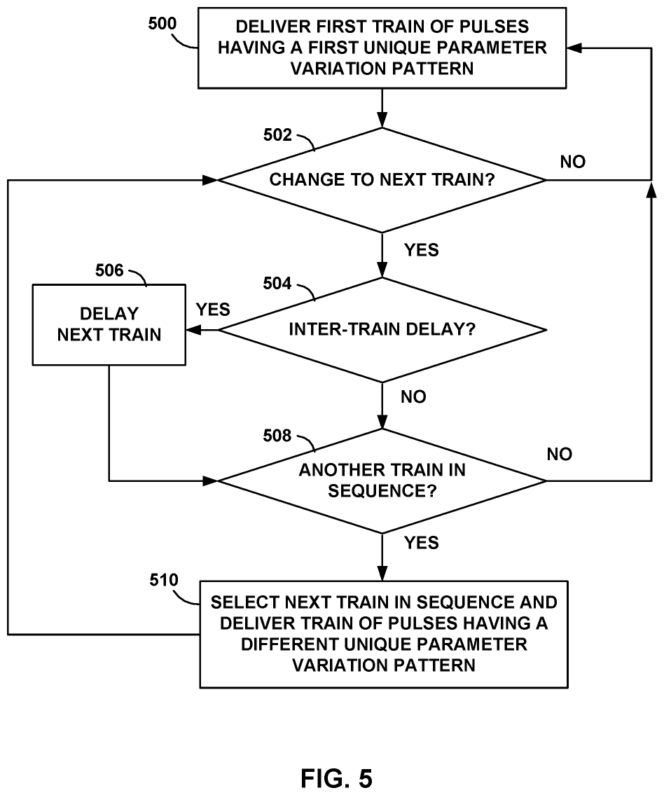

FIG. 5 is a flow diagram illustrating an example technique for delivering electrical stimulation having a sequence of different trains of pulses. FIG. 5 is a flow diagram illustrating an example technique of delivering a sequence of different pulse trains having unique parameter variation patterns. The technique of FIG. 5 will be described with respect to processing circuitry 32 of IMD 14 within system 10 of FIG. 1. In other examples, however, the technique of FIG. 5 may be used with other devices within system 10 or a system other than system 10 of FIG. 1, such as by patient programmer 22 or a networked server (e.g., could computing system) or other device and/or system. In other examples, an external neurostimulator may perform the functions of FIG. 5 instead of an implantable device such as IMD 14.

As shown in the example of FIG. 5, processing circuitry 32 of IMD 14 delivers a first train of pulses having a first unique parameter variation pattern (500). If the pulse train is not ending (“NO” branch of block 502), then processing circuitry 32 continues to deliver the first train of pulses (500). Processing circuitry 32 may determine which trains need to be delivered based on instructions stored and tracking the delivery of pulses over time and/or numbers of pulses. If processing circuitry 32 determines that the next train is to begin (“YES” branch of block 502), processing circuitry 32 determines if there is to be an inter-train delay (504). If there is an inter-train delay (“YES” branch of block 504), processing circuitry 32 performs a delay for the next train for a certain period of time (506) and then determines if there is another train in the sequence (508).

If there is no inter-train delay (“NO” branch of block 504), processing circuitry 32 determines if there is another train to be delivered in the sequence (508). If there is another train in the sequence, such as a second train of pulses having a second unique parameter variation pattern (“YES” branch of block 508), processing circuitry 32 selects the next train in the sequence and controls IMD 14 to deliver that train of pulses having a different unique parameter variation pattern (510). Processing circuitry 32 then determines whether or not to change to another train (502). If there is no additional train in the sequence (“NO” branch of block 508), processing circuitry 32 controls IMD 14 to deliver the first train of pulses again (500) to repeat the sequence.

In some examples, processing circuitry 32 may automatically make adjustments to the delivery of the pulse trains within the sequence or when to deliver the sequence. For example, processing circuitry 32 may cycle stimulation by turning on and off the delivery of the sequence of pulse trains. Processing circuitry 32 may deliver stimulation according to the sequence for a certain period of time or a certain number of sequences which may establish a duty cycle for stimulation. In some examples, processing circuitry 32 may adjust the cycling of stimulation. For example, processing circuitry 32 may gradually increase the inter-sequence delay and/or the inter-train delay until the patient, a clinician, or sensed information indicates that the stimulation therapy is no longer effective. The patient may provide input to programmer 20, for example, to indicate that stimulation is not effective or request a change to stimulation that may indicate that stimulation is not effective, such as requesting an increase to amplitude. Then, processing circuitry 32 may revert to a previous inter-sequence delay and/or the inter-train delay at which stimulation remained effective at treating the symptoms of the patient. By reducing the amount of time stimulation is delivered, processing circuitry 32 may reduce the power consumption of therapy and increase the battery longevity of IMD 14. In addition, reducing power consumption may also increase the time between recharge sessions and/or reduce the duration of a recharge session which may reduce the amount of time the patient needs to recharge IMD 14.

In some examples, processing circuitry 32 may adjust the sequence or pulse trains within a sequence according to various information. For example, IMD 14 may monitor one or more sensed signals (e.g., bladder volume, bladder pressure, patient activity, time between voids, bladder/bowel leaks etc.) for indications of therapy efficacy. In response to determining that a therapy efficacy is declining during delivery of a pulse train with a respective parameter variation pattern, processing circuitry 32 may remove that pulse train from the sequence. In some examples, processing circuitry 32 may add a different pulse train with a different parameter variation pattern in the place of the dropped pulse train. Alternatively, processing circuitry 32 may duplicate a pulse train that appears to be effective. In this manner, processing circuitry 32 may skip, remove, or add pulse trains in response to indications that a pulse train is effective or ineffective. Processing circuitry 32 may add or remove different pulse trains to the sequence as a function of time, such as inserting new pulse trains with different parameter variation patterns that may reduce power consumption or treat different areas of tissue to further reduce the risk of habituation to stimulation over time.

Additionally, or alternatively, processing circuitry 32 may adjust the sequence or pulse trains within a sequence in response to user input. For example, processing circuitry 32 (via programmer 22 or at the instruction of programmer 22) may receive a request to skip one or more trains of the plurality of trains of electrical stimulation pulses within the sequence. The request may be received via a user interface of programmer 22, for example. The user may explicitly identify the pulse train, or the pulse train being delivered at the time the request to skip was received may be identified by programmer 22 or IMD 14. Responsive to receiving the request to skip the one or more trains, processing circuitry 32 may remove the one or more trains from the sequence to generate a new sequence of trains of electrical stimulation pulses. Processing circuitry 32 may then control the stimulation circuitry to deliver the new sequence of pulse trains of electrical stimulation pulses. In some examples, processing circuitry 32 may be configured to generate the new sequence of trains by adding a new train of electrical stimulation pulses that includes pulses varied according to a parameter variation pattern unique from parameter variation patterns of other pulse trains already in the sequence prior to removing the other pulse train.

Although FIG. 5 was described with respect to IMD 14, and the processing circuitry of IMD 14, in other examples, one or more additional devices, components, processing circuitry, or any combination thereof, may perform one or more steps of any of the techniques described herein. Moreover, the techniques of FIG. 5 may be implemented in any order, may be implemented simultaneously, may have one or more steps added and/or deleted, and may be implemented in combination with one another.

The following examples are described herein.

Example 1: A system includes processing circuitry configured to: control stimulation circuitry to deliver a sequence of a plurality of trains of electrical stimulation pulses, wherein each train of the plurality of trains of electrical stimulation pulses comprises respective pulses at least partially defined by a unique parameter variation pattern of a plurality of parameter variation patterns; and control the stimulation circuitry to repeatedly deliver the sequence of the plurality of trains of electrical stimulation pulses.

Example 2: The system of example 1, wherein at least one parameter variation pattern of the plurality of parameter variation patterns defines a varied pulse width of at least some pulses of a train of electrical stimulation pulses.

Example 3: The system of any of examples 1 and 2, wherein at least one parameter variation pattern of the plurality of parameter variation patterns defines a varied polarity between at least some pulses of a train of electrical stimulation pulses.

Example 4: The system of any of examples 1 through 3, wherein at least one parameter variation pattern of the plurality of parameter variation patterns defines a varied frequency of at least some pulses of a train of electrical stimulation pulses.

Example 5: The system of any of examples 1 through 4, wherein at least one parameter variation pattern of the plurality of parameter variation patterns defines a varied amplitude of at least some pulses of a train of electrical stimulation pulses.

Example 6: The system of any of examples 1 through 5, wherein at least one parameter variation pattern of the plurality of parameter variation patterns defines a varied electrode combination of at least some pulses of a train of electrical stimulation pulses.

Example 7: The system of any of examples 1 through 6, wherein: the plurality of parameter variation patterns comprises a first unique parameter variation pattern defining a first parameter that is varied and a second unique parameter variation pattern defining a second parameter that is varied, the first parameter being different than the second parameter; and the sequence comprises a first train of the plurality of trains of electrical stimulation pulses comprising pulses at least partially defined by the first unique parameter variation pattern and a second train of the plurality of trains of electrical stimulation pulses comprising pulses at least partially defined by the second unique parameter variation pattern.

Example 8: The system of any of examples 1 through 7, wherein the sequence is a first sequence, and wherein the processing circuitry is configured to: receive a request to skip one or more trains of the plurality of trains of electrical stimulation pulses; responsive to receiving the request to skip the one or more trains, remove the one or more trains from the first sequence to generate a second sequence of trains of electrical stimulation pulses; and control the stimulation circuitry to deliver the second sequence of trains of electrical stimulation pulses.

Example 9: The system of example 7, wherein the processing circuitry is configured to generate the second sequence of trains by adding a new train of electrical stimulation pulses comprising pulses varied according to a parameter variation pattern unique from parameter variation patterns that at least partially define the variation of respective pulses of trains of the second sequence of trains.

Example 10: The system of any of examples 1 through 9, wherein the processing circuitry is configured to separate consecutive trains of the plurality of trains of electrical stimulation pulses within the sequence by an inter-train delay period.

Example 11: The system of any of examples 1 through 10, wherein the processing circuitry is configured to separate consecutive deliveries of the sequence by an inter-sequence delay period.

Example 12: The system of any of examples 1 through 11, wherein the sequence of the plurality of trains of electrical stimulation pulses comprises at least four trains of electrical stimulation pulses, each train of the four trains of electrical stimulation pulses comprising respective pulses that are varied according to respective unique parameter variation patterns of the plurality of parameter variation patterns.

Example 13: The system of any of examples 1 through 12, further comprising an implantable medical device comprising the processing circuitry and the stimulation circuitry.

Example 14: A method comprises controlling, by processing circuitry, stimulation circuitry to deliver a sequence of a plurality of trains of electrical stimulation pulses, wherein each train of the plurality of trains of electrical stimulation pulses comprises respective pulses at least partially defined by a unique parameter variation pattern of a plurality of parameter variation patterns; and controlling, by the processing circuitry, the stimulation circuitry to repeatedly deliver the sequence(s) of the plurality of trains of electrical stimulation pulses.

Example 15: The method of example 14, wherein at least one parameter variation pattern of the plurality of parameter variation patterns defines a varied pulse width of at least some pulses of a train of electrical stimulation pulses.

Example 16: The method of any of examples 14 and 15, wherein at least one parameter variation pattern of the plurality of parameter variation patterns defines a varied polarity between at least some pulses of a train of electrical stimulation pulses.

Example 17: The method of any of examples 13 through 16, wherein at least one parameter variation pattern of the plurality of parameter variation patterns defines a varied frequency of at least some pulses of a train of electrical stimulation pulses.

Example 18: The method of any of examples 13 through 17, wherein at least one parameter variation pattern of the plurality of parameter variation patterns defines a varied amplitude of at least some pulses of a train of electrical stimulation pulses.

Example 19: The method of any of examples 13 through 18, wherein at least one parameter variation pattern of the plurality of parameter variation patterns defines a varied electrode combination of at least some pulses of a train of electrical stimulation pulses.

Example 20: The method of any of examples 13 through 19, wherein: the plurality of parameter variation patterns comprises a first unique parameter variation pattern defining a first parameter that is varied and a second unique parameter variation pattern defining a second parameter that is varied, the first parameter being different than the second parameter; and the sequence comprises a first train of the plurality of trains of electrical stimulation pulses comprising pulses at least partially defined by the first unique parameter variation pattern and a second train of the plurality of trains of electrical stimulation pulses comprising pulses at least partially defined by the second unique parameter variation pattern.

Example 21: The method of any of examples 13 through 20, wherein the sequence is a first sequence, and wherein the method further comprises: receiving a request to skip one or more trains of the plurality of trains of electrical stimulation pulses; responsive to receiving the request to skip the one or more trains, removing the one or more trains from the first sequence to generate a second sequence of trains of electrical stimulation pulses; and controlling the stimulation circuitry to generate the second sequence of trains of electrical stimulation pulses.

Example 22: The method of example 21, wherein generating the second sequence of trains comprises adding a new train of electrical stimulation pulses comprising pulses varied according to a parameter variation pattern unique from parameter variation patterns that at least partially define the variation of respective pulses of trains of the second sequence of trains.

Example 23: The method of any of examples 13 through 22, further comprising separating consecutive trains of the plurality of trains of electrical stimulation pulses within the sequence by an inter-train delay period.

Example 24: The method of any of examples 13 through 23, further comprising separating consecutive deliveries of the sequence by an inter-sequence delay period.

Example 25: The method of any of examples 13 through 24, wherein the sequence of the plurality of trains of electrical stimulation pulses comprises at least four trains of electrical stimulation pulses, each train of the four trains of electrical stimulation pulses comprising respective pulses that are varied according to respective unique parameter variation patterns of the plurality of parameter variation patterns.

Example 26: A computer readable storage medium comprising instructions that, when executed, control processing circuitry to control stimulation circuitry to deliver a sequence of a plurality of trains of electrical stimulation pulses, wherein each train of the plurality of trains of electrical stimulation pulses comprises respective pulses at least partially defined by a unique parameter variation pattern of a plurality of parameter variation patterns; and control the stimulation circuitry to repeatedly deliver the sequence of the plurality of trains of electrical stimulation pulses.

The techniques described in this disclosure, including those attributed to system 10, IMD 14, patient programmer 22, and clinician programmer 20, and various constituent components, may be implemented, at least in part, in hardware, software, firmware or any combination thereof. For example, various aspects of the techniques may be implemented within one or more processors or processing circuitry, including one or more microprocessors, DSPs, ASICs, FPGAs, or any other equivalent integrated or discrete logic circuitry, as well as any combinations of such components, remote servers, cloud servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques or processes described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), ferroelectric random access memory (FRAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices. In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

Various examples have been described herein. Any combination of the described operations or functions is contemplated. These and other examples are within the scope of the following claims.

What is claimed is:

1. A system comprising:
processing circuitry configured to:
control stimulation circuitry to deliver a sequence of a plurality of trains of electrical stimulation pulses, wherein each train of the plurality of trains of electrical stimulation pulses comprises respective pulses at least partially defined by a unique parameter variation pattern of a plurality of parameter variation patterns; and
control the stimulation circuitry to repeatedly deliver the sequence of the plurality of trains of electrical stimulation pulses.

2. The system of claim 1, wherein at least one parameter variation pattern of the plurality of parameter variation patterns defines a varied pulse width of at least some pulses of a train of electrical stimulation pulses.

3. The system of claim 1, wherein at least one parameter variation pattern of the plurality of parameter variation patterns defines a varied polarity between at least some pulses of a train of electrical stimulation pulses.

4. The system of claim 1, wherein at least one parameter variation pattern of the plurality of parameter variation patterns defines a varied frequency of at least some pulses of a train of electrical stimulation pulses.

5. The system of claim 1, wherein at least one parameter variation pattern of the plurality of parameter variation patterns defines a varied amplitude of at least some pulses of a train of electrical stimulation pulses.

6. The system of claim 1, wherein at least one parameter variation pattern of the plurality of parameter variation patterns defines a varied electrode combination of at least some pulses of a train of electrical stimulation pulses.

7. The system of claim 1, wherein:
the plurality of parameter variation patterns comprises a first unique parameter variation pattern defining a first parameter that is varied and a second unique parameter variation pattern defining a second parameter that is varied, the first parameter being different than the second parameter; and
the sequence comprises a first train of the plurality of trains of electrical stimulation pulses comprising pulses at least partially defined by the first unique parameter variation pattern and a second train of the plurality of trains of electrical stimulation pulses comprising pulses at least partially defined by the second unique parameter variation pattern.

8. The system of claim 1, wherein the sequence is a first sequence, and wherein the processing circuitry is configured to:
receive a request to skip one or more trains of the plurality of trains of electrical stimulation pulses;
responsive to receiving the request to skip the one or more trains, remove the one or more trains from the first sequence to generate a second sequence of trains of electrical stimulation pulses; and
control the stimulation circuitry to deliver the second sequence of trains of electrical stimulation pulses.

9. The system of claim 7, wherein the processing circuitry is configured to generate the second sequence of trains by adding a new train of electrical stimulation pulses comprising pulses varied according to a parameter variation pattern unique from parameter variation patterns that at least partially define the variation of respective pulses of trains of the second sequence of trains.

10. The system of claim 1, wherein the processing circuitry is configured to separate consecutive trains of the plurality of trains of electrical stimulation pulses within the sequence by an inter-train delay period.

11. The system of claim 1, wherein the processing circuitry is configured to separate consecutive deliveries of the sequence by an inter-sequence delay period.

12. The system of claim 1, wherein the sequence of the plurality of trains of electrical stimulation pulses comprises at least four trains of electrical stimulation pulses, each train of the four trains of electrical stimulation pulses comprising respective pulses that are varied according to respective unique parameter variation patterns of the plurality of parameter variation patterns.

13. The system of claim 1, further comprising an implantable medical device comprising the processing circuitry and the stimulation circuitry.

14. A method comprising:
controlling, by processing circuitry, stimulation circuitry to deliver a sequence of a plurality of trains of electrical stimulation pulses, wherein each train of the plurality of trains of electrical stimulation pulses comprises respective pulses at least partially defined by a unique parameter variation pattern of a plurality of parameter variation patterns; and
controlling, by the processing circuitry, the stimulation circuitry to repeatedly deliver the sequence(s) of the plurality of trains of electrical stimulation pulses.

15. The method of claim 14, wherein at least one parameter variation pattern of the plurality of parameter variation patterns defines a varied pulse width of at least some pulses of a train of electrical stimulation pulses.

16. The method of claim 14, wherein at least one parameter variation pattern of the plurality of parameter variation patterns defines a varied polarity between at least some pulses of a train of electrical stimulation pulses.

17. The method of claim 14, wherein at least one parameter variation pattern of the plurality of parameter variation patterns defines a varied frequency of at least some pulses of a train of electrical stimulation pulses.

18. The method of claim 14, wherein at least one parameter variation pattern of the plurality of parameter variation patterns defines a varied amplitude of at least some pulses of a train of electrical stimulation pulses.

19. The method of claim 14, wherein:
the plurality of parameter variation patterns comprises a first unique parameter variation pattern defining a first parameter that is varied and a second unique parameter variation pattern defining a second parameter that is varied, the first parameter being different than the second parameter; and
the sequence comprises a first train of the plurality of trains of electrical stimulation pulses comprising pulses at least partially defined by the first unique parameter variation pattern and a second train of the plurality of trains of electrical stimulation pulses comprising pulses at least partially defined by the second unique parameter variation pattern.

20. A computer readable storage medium comprising instructions that, when executed, causes processing circuitry to:
control stimulation circuitry to deliver a sequence of a plurality of trains of electrical stimulation pulses, wherein each train of the plurality of trains of electrical stimulation pulses comprises respective pulses at least partially defined by a unique parameter variation pattern of a plurality of parameter variation patterns; and
control the stimulation circuitry to repeatedly deliver the sequence of the plurality of trains of electrical stimulation pulses.
